(19) United States
(12) Patent Application Publication
(10) Pub. No.: US 2026/0337221 A1
(43) Pub. Date: Oct. 29, 2026

(54) Compositions and Methods for Selective Labeling of N-Alkylated Imidazole Containing Compounds and Peptides

(71) Applicant: Emory University, Atlanta, GA (US)

(72) Inventors: Monika Raj, Atlanta, GA (US); Patricia Rodriguez, Atlanta, GA (US)

(21) Appl. No.: 19/105,683

(22) PCT Filed: Aug. 23, 2023
(86) PCT No.: PCT/US2023/030897

§ 371 Date: Feb. 21, 2025

Related U.S. Application Data
(60) Provisional application No. 63/400,155, filed on Aug. 23, 2022.

(51) Int. Cl. C07K 1/13; C07K 5/097

(52) U.S. Cl. CPC C07K 1/13; C07K 5/0821

(57) ABSTRACT

This disclosure relates to compositions and methods useful for selective labeling of compounds containing an N-alkylated imidazole ring. In certain embodiments, this disclosure relates to methods of forming an oxime compound, comprising contacting a compound containing an N-alkyl imidazole ring with alpha alkynyl ketone providing an N-alkyl formamide derivative and contacting the N-alkyl formamide derivative with a hydroxy amine providing an oxime compound. In certain embodiments, the N-alkyl imidazole ring is methyl histidine present in peptides or other biological molecules.

### CROSS-REFERENCE TO RELATED APPLICATIONS

This application claims the benefit of U.S. Provisional Application No. 63/400,155 filed Aug. 23, 2022. The entirety of this application is hereby incorporated by reference for all purposes.

### BACKGROUND

Amino acid sequencing of naturally occurring proteins and antibodies may be accomplished by reconstructing segments of degraded and labeled protein fragments including the utilization of unique amino acid markers and size exclusion chromatography. Mass spectrometry can be helpful to identify sequences of smaller segments, but the technique becomes inherently less reliable as the protein segments become larger. Thus, there is a need to identify improved methods for identifying and sequencing proteins using selective amino acid labels.

Histidine is an amino acid containing a side chain with an imidazole ring. Nitrogen atoms in the imidazole ring are sometimes converted to N-methylated derivatives by post-translational modifications (PTMs). Histidine methylation of proteins is reported to be involved in various biological processes. Aberrant methylated histidine is linked to certain disorders including cancer. Inhibitors of human histone transmethylase EZH2 are reported to be useful as anticancer agents. Limited histidine methylation sites are known when compared to other PTMs because chemical technologies for detecting histidine methylation are also limited. The addition of small methyl groups leads to the negligible alteration in physicochemical properties of a protein making detection challenging. Thus, there is a need to identify improved methods for identifying histidine methylation.

Zinellu et al. report quantification of histidine, 1-methylhistidine and 3-methylhistidine in plasma and urine by capillary electrophoresis UV-detection. J Sep Sci. 2010, 33 (23-24): 3781-5.

Tang et al. report a one-step azolation strategy for site- and chemo-selective labeling of proteins with mass-sensitive probes. Angew Chem Int Ed, 2021, 60, 1797-1805.

Tang et al. report tunable amine-reactive electrophiles for selective profiling of lysine. Angew Chem Int Ed, 2022, 61, e202112107.

References cited herein are not an admission of prior art.

### SUMMARY

This disclosure relates to compositions and methods for selective labeling of compounds containing N-alkylated imidazole rings such as methyl histidine. In certain embodiments, this disclosure relates to methods of forming an oxime compound comprising contacting a compound containing an N-alkyl imidazole ring with alpha alkynyl ketone providing an N-methyl formamide derivative and contacting the N-methyl formamide derivative with a hydroxy amine providing an oxime compound. In certain embodiments, the N-alkyl imidazole ring is methyl histidine present in peptides or other biological molecules.

In certain embodiments, the N-alkyl imidazole ring is methyl imidazole contained in a peptide, nucleic acid, nucleobase polymer, compound, polysaccharide, or other biological molecule.

In certain embodiments, this disclosure relates to methods of forming oxime labeled peptides or compounds comprising contacting a compound containing an N-alkylated imidazole ring such as methyl histidine with an alpha alkynyl ketone, 1,3-diphenylprop-2-yn-1-one, or derivative providing a formamide and chalcone derivative and contacting the formamide and chalcone derivative with a labeled hydroxy amine providing an oxime labeled peptide or compound. In certain embodiments, the label is an aromatic molecule, a fluorescent dye, a ligand, biotin, a receptor, an antigen, or an antibody.

In certain embodiments, this disclosure relates to compounds, compositions, and materials comprising or coated with compounds disclosed herein.

### BRIEF DESCRIPTION OF THE SEVERAL VIEWS OF THE DRAWINGS

FIG. 1 illustrates chemical names for compounds and intermediates of this disclosure.

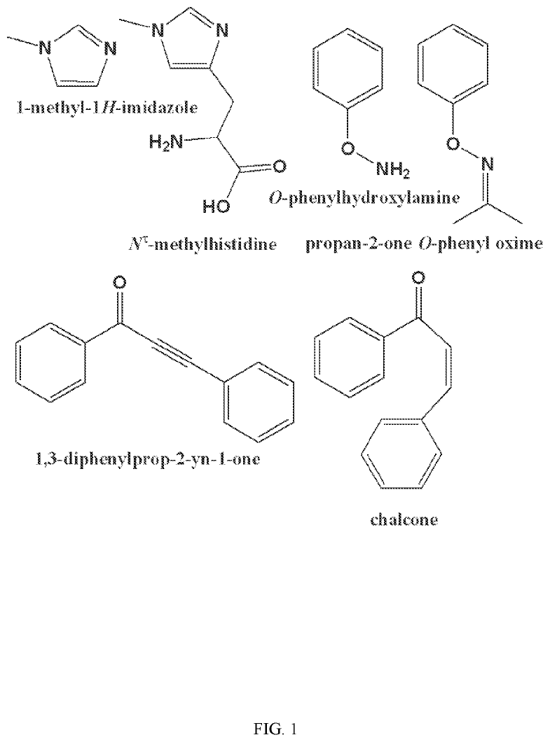

FIG. 2 illustrates a method of labeling methyl histidine by contacting a methyl histidine containing peptide with 1,3-diphenylprop-2-yn-1-one providing a formamide and chalcone derivative and contacting the formamide and chalcone derivative with a O-phenyl hydroxy amine providing an O-phenyl oxime labeled peptide with an affinity tag or fluorophore. The unmethylated histidine chalcone derivative reverts to the natural histidine amino acid in the final acid conditions. Thus, the overall process is selective for labeling methyl histidine.

FIG. 3 illustrates a method wherein a hydroxy amine compound is used to label a peptide with the formamide and chalcone derivative providing an oxime compound with an alkynyl group that allows for reaction with solid surface coated with an azide providing a heterocyclic linking group attached the solid surface due to an azide-alkyne cycloaddition reaction.

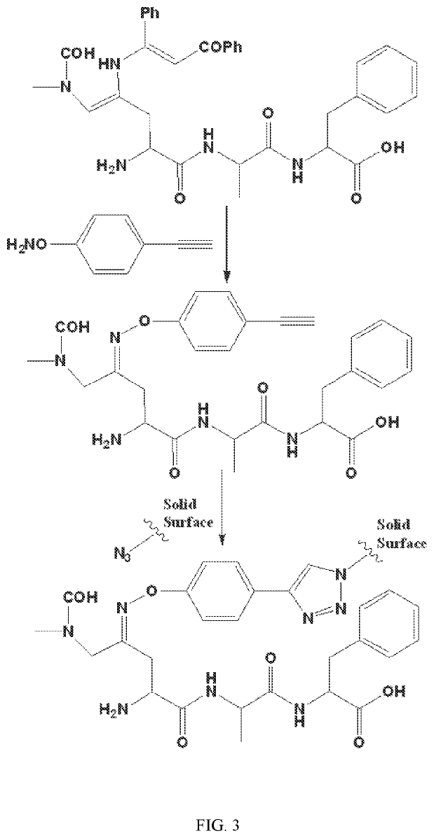

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure indicates that it is an example and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “protein,” “peptide,” and “polypeptide” refer to compounds comprising amino acids joined via peptide bonds and are used interchangeably. As used herein, an “amino acid sequence” refers to an amino acid sequence of a protein molecule. An “amino acid sequence” can be deduced from the nucleic acid sequence encoding the protein. However, terms such as “peptide” or “protein” are not meant to limit be limited to natural amino acids. The term includes naturally and non-naturally derived material optionally having naturally or non-naturally occurring amino acids and modifications such as, substitutions, glycosylations, and addition of hydrophilic or lipophilic moieties. In certain embodiments, the protein/peptide/polypeptide comprises more than three, four, five, six, seven, eight, nine, or ten amino acids.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may contain DNA or RNA or a combination of DNA or RNA nucleotides or may be single or double stranded or both, e.g., they may contain overhangs, hairpins, bends, etc. Nucleobase polymers may contain naturally occurring or synthetically modified bases and backbones.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal) Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH—CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, and aromatic groups.

The term “specific binding agent” refers to a molecule, such as a proteinaceous molecule, that binds a target molecule with a greater affinity than other random molecules or proteins. Examples of specific binding agents include antibodies that bind an epitope of an antigen or a receptor which binds a ligand. “Specifically binds” refers to the ability of a specific binding agent (such as a ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250, or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

Hydrophilic polymers contain polar or charged functional groups, rendering them soluble in water. Examples include polyethylene glycol, polylactides, polyglycolide, poly(ε-caprolactone), poly(2-methoxyethyl acrylate), poly(tetrahydrofurfuryl acrylate), poly(2-methacryloyloxyethyl phosphorylcholine), poly(p-dioxanone), poly(serine methacrylate), poly [oligo (ethylene glycol) vinyl ether], poly {[2-(methacryloyloxy)ethyl], copolymers of ethylene glycol and propylene glycol, poly(oxyethylated polyol), poly(olefmic alcohol), poly(vinylpyrrolidone), poly(hydroxyalkylmethacrylamide), poly(hydroxyalkylmethacrylate), poly(saccharides), poly(alpha-hydroxy acid), and poly(vinyl alcohol). “PEG,” “polyethylene glycol” and “poly(ethylene glycol)” refers to water-soluble poly(ethylene oxide). In certain embodiments, PEGs comprise the following structure “—(OCH₂CH₂)ₙ-” where (n) is 2 to 4000.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. A derivative may be a polypeptide variant. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O) NRaNRb, —NRₐC(═O)OR_{b}, —NRₐSO₂R_{b}, —C(═O)Rₐ, —C(═O)ORₐ, —C(═O) NRₐR_{b}, —OC(═O) NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Haloalkyl” refers to an alkyl group wherein one or more or all of the hydrogens are substituted with a halogen(s), e.g., —CH₂CH₂Cl or —CF₃.

“Alkylthio” refers to an alkyl group as defined above with the indicated number of carbon atoms attached through a sulfur bridge. An example of an alkylthio is methylthio, (e.g., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (e.g., —NH—CH₃).

“Alkanoyl” refers to an alkyl as defined above with the indicated number of carbon atoms attached through a carbonyl bride (e.g., —(C═O)alkyl).

“Alkylthio” refers to an alkyl group as defined above with the indicated number of carbon atoms attached through a sulfur bridge. An example of an alkylthio is methylthio, (e.g., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Carboxyl” refers to a carboxylic acid and a “carboxyl ester” refers to an ester of the acid (e.g., —(C═O)O-alkyl).

“Alkylamino” refers an alkyl group as defined above with the indicated number of carbon atoms attached through an amino bridge. An example of an alkylamino is methylamino, (e.g., —NH—CH₃).

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

The term, “chalcone” refers to alpha, beta-unsaturated ketones substituted with optionally substituted aryl or optionally substituted heteroaryl, such as pyridinyl. The compound 1,3-diphenylprop-2-en-1-one is a chalcone. A chalcone may be in the “E” or “Z” configuration. Chalcones may be produced by the condensation reaction of an aldehyde and a ketone, e.g., aryl or heteroaryl aldehyde, e.g., benzaldehyde, and aryl or heteroaryl ketone, e.g., acetophenone. Chalcones are found in natural products, e.g., as intermediates in the production of flavonoids.

### Methods and Compositions for Detecting Methyl Imidazole Groups on Compounds, Peptides and Other Biological Molecules Containing Methyl Histidine

Disclosed herein are methods and composition for tagging methyl histidine leading to the formation of a stable product allowing for identification and characterization of histidine methylation sites globally, independent of the peptide sequence.

In certain embodiments, this disclosure relates to compositions and methods for selective labeling of compounds containing N-alkylated imidazole ring, N-methyl imidazole, or methyl histidine. In certain embodiments, the N-alkylated imidazole ring is methyl histidine present in peptides or other biological molecules such as nucleic acids or polysaccharides. In certain embodiments, the N-alkylated imidazole ring is present on aliphatic or aromatic compounds.

In certain embodiments, this disclosure relates to methods of forming oxime labeled peptides or compounds comprising contacting a compound containing an N-alkylated imidazole ring such as methyl histidine with an alpha alkynyl ketone providing a formamide and chalcone derivative and contacting the formamide and chalcone derivative with a labeled hydroxy amine providing an oxime labeled peptide or compound. In certain embodiments, the label is an aromatic molecule, a fluorescent dye, a ligand, biotin, a receptor, an antibody, or an antigen.

In certain embodiments, this disclosure relates to methods of forming O-aryl oxime labeled peptides or compounds, comprising contacting a compound containing an N-alkylated imidazole ring such as methyl histidine with an alpha alkynyl ketone providing a formamide and chalcone derivative and contacting the formamide and chalcone derivative with a labeled O-aryl hydroxy amine providing an O-aryl oxime labeled peptide or compound. In certain embodiments, the label is an aromatic molecule, a fluorescent dye, a ligand, biotin, a receptor, an antibody, or an antigen.

In certain embodiments, this disclosure relates to methods of forming an O-phenyl oxime compound comprising contacting a compound containing an N-alkylated imidazole ring with an alpha alkynyl ketone, 1,3-diphenylprop-2-yn-1-one, or derivative providing an N-methyl formamide derivative and contacting the N-methyl formamide derivative with an O-phenyl hydroxy amine providing an O-phenyl oxime compound.

In certain embodiments, this disclosure relates to methods of forming O-heteroaryl oxime labeled peptides or compounds comprising contacting a compound containing an N-alkylated imidazole ring such as methyl histidine with an alpha alkynyl ketone providing a formamide and chalcone derivative and contacting the formamide and chalcone derivative with a labeled O-heteroaryl hydroxy amine providing an O-heteroaryl oxime labeled peptide or compound. In certain embodiments, the label is an aromatic molecule, a fluorescent dye, a ligand, biotin, a receptor, an antibody, or an antigen.

In certain embodiments, this disclosure relates to methods of forming O-alkyl oxime labeled peptides or compounds comprising contacting a compound containing an N-alkylated imidazole ring such as methyl histidine with an alpha alkynyl ketone providing a formamide and chalcone derivative and contacting the formamide and chalcone derivative with a labeled O-alkyl hydroxy amine providing an O-alkyl oxime labeled peptide or compound. In certain embodiments, the label is an aromatic molecule, a fluorescent dye, a ligand, biotin, a receptor, an antibody, or an antigen.

In certain embodiments, the compound containing an N-alkylated imidazole ring is methyl histidine or peptide comprising the same.

In certain embodiments, contacting a compound containing an N-alkylated imidazole ring with an alpha alkynyl ketone, 1,3-diphenylprop-2-yn-1-one or derivative is in a solution of acetonitrile and water.

In certain embodiments, the solution of acetonitrile and water at a pH of between 6 and 8.

In certain embodiments, the solution of acetonitrile and water at a temperature of between 50 degrees C. and 70 degrees C.

In certain embodiments, contacting the formamide derivative with a hydroxy amine is in an aqueous solution at a pH of less than 2.

In certain embodiments, contacting the formamide derivative with a hydroxy amine is at a temperature of between 40 degrees C. and 60 degrees C.

In certain embodiments, the hydroxy amine comprises a label. In certain embodiments, the label is an aromatic molecule, a fluorescent dye, a ligand, biotin, a receptor, an antibody, or an antigen.

In certain embodiments, this disclosure relates to compounds, compositions, and materials comprising or coated with compounds disclosed herein.

In certain embodiments, this disclosure relates to methods and compositions for labeling, isolating, detecting, measuring, and/or purifying compounds containing N-alkylated imidazole ring or methyl histidine made by processes disclosed herein. In certain embodiments, this disclosure relates to methods of labeling, isolating, detecting, measuring, purifying, quantifying, and amino acid sequencing compounds comprising methyl histidine or a peptide containing the same from a sample optionally utilizing solid supports. In certain embodiments, the sample is from a human subject. In certain embodiments, the sample is a blood sample, urine sample, stool sample, saliva sample, or sputum sample.

In certain embodiments, the peptide or compound for labeling having a N-alkylated imidazole ring or methyl histidine is a synthetic amino acid, a nucleobase, nucleic acid, DNA, RNA, nucleobase polymer, alkyl, aryl, carbocyclyl, or heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbonyl ester, carbamoyl, aryl, carbocyclyl or heterocyclyl, or N-substituted carbamoyl group, which is optionally further substituted or conjugated to a label or a solid support through a linking group.

In certain embodiments, for any of the methods disclosed herein the methods further comprise determining the molecular weight or exact mass of the peptide, compound, oxime labeled peptide, or oxime labeled compound.

In certain embodiments, for any of the methods disclosed herein reactions are at between about 50 and 70 degrees Celsius. In certain embodiments, for any of the methods disclosed herein reactions are at between about 40 and 60 degrees Celsius. In certain embodiments, for any of the methods disclosed herein reactions are at between about 40 and 70 degrees Celsius. In certain embodiments, for any of the methods disclosed herein reactions are at or about room temperature or slightly above room temperature, e.g., between 10 and 40 degrees Celsius.

In certain embodiments, this disclosure relates to methods of contacting a compound or protein containing a N-alkylated imidazole ring or methyl histidine wherein the compound or protein containing a N-alkylated imidazole ring or methyl histidine or peptide containing the same is in a sample, such as a biological sample, (e.g., cell, tissue, etc.) or environmental sample. Biological samples may be obtained from animals (including humans) and encompass fluids, blood, solids, tissues, and gases. Environmental samples include environmental material such as surface matter, soil, water, and industrial samples.

In certain embodiments, for any of the method disclosed herein, the conditions for providing an oxime labeled peptide or compound that does not result in the labeling of unmethylated histidine, amides and/or terminal amines, including primary, secondary, nor tertiary amines, in the peptide, compound, or sample.

In certain embodiments, the oxime labeled peptide or compound is an alkynyl labeled compound such as benzene para substituted with an alkynyl group.

In certain embodiments, the oxime labeled peptide or compound is further substituted with a label.

In certain embodiments, the label is biotin, aromatic molecule, a fluorescent dye, a second alkynyl group, ligand, biotin, receptor, antibody, or antigen.

In certain embodiments, the methods further comprise contacting the alkynyl labeled compound with a solid surface conjugated to a triazene under conditions such that a triazole compound is conjugated to the solid surface.

In certain embodiments, methods further comprise contacting the triazole compound conjugated to the solid surface with an acid solution such that the purified compound is cleaved from the solid surface providing purified compound.

In certain embodiments, the label is a ligand providing ligand labeled compound or peptide. In certain embodiments, the method further comprises contacting the ligand labeled or peptide with a solid surface conjugated to a receptor under conditions such that the ligand labeled compound or peptide is conjugated to the solid surface.

In certain embodiments, the label is biotin providing biotin labeled or peptide. In certain embodiments, the method further comprises contacting the biotin labeled compound or peptide with a solid surface conjugated to avidin or streptavidin under conditions such that the biotin labeled compound or peptide is conjugated to the solid surface.

In certain embodiments, this disclosure relates to methods of forming oxime labeled peptides or compounds, comprising contacting a compound containing an N-alkylated imidazole ring such as methyl histidine with an alpha alkynyl ketone, 1,3-diphenylprop-2-yn-1-one, or derivative providing a formamide and chalcone derivative and contacting the formamide and chalcone derivative with a labeled hydroxy amine providing an oxime labeled peptide or compound.

In certain embodiments, the labeled hydroxy amine compound has the following formula,

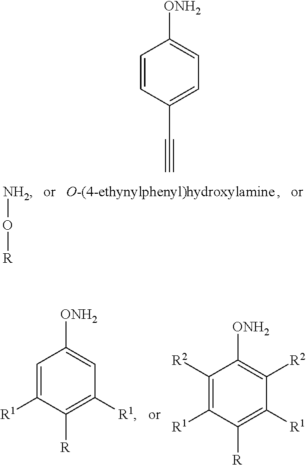

or derivatives thereof, wherein R is optionally substituted with a label or conjugated to a solid support through a linking group. In certain embodiments, the linking group is a hydrophilic polymer. In certain embodiments, R is alkyl, aryl, carbocyclyl, or heterocyclyl, wherein R is optionally substituted with one or more substituents, or conjugated to a label or a solid support through a linking group. In certain embodiments, R¹ is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R¹ is optionally substituted. In certain embodiments, R² is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R² is optionally substituted. In certain embodiments, R, R¹ or R² are individually and independently at each occurrence hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R, R¹ or R² are optionally substituted e.g., conjugated to a label or a solid support through a linking group, or R¹ or R, or R¹ or R², and the attached atoms together form an aromatic or non-aromatic ring optionally substituted e.g., conjugated to a label or a solid support through a linking group.

In certain embodiments, R, R¹ or R² is a linking group comprising an alkynyl group or R is —(C═O)NHR³, —(C—O)OR³, —(C—O)SR³, or —(C═O)R³ wherein R³ is an alkynyl group. In certain embodiments, R, R¹ or R² is a linking group comprising an alkynyl group or R, R¹ or R² is —(C—O)NHR³, —(C═O)OR³, —(C═O)SR³, or —(C—O)R³, wherein R³ is an alkynyl group.

In certain embodiments, this disclosure relates to methods of purifying a compound comprising an N-alkylated imidazole ring such as methyl histidine in a sample comprising contacting the sample with an alpha alkynyl ketone, 1,3-diphenylprop-2-yn-1-one, or derivative providing a formamide and chalcone derivative; contacting the formamide and chalcone derivative with a labeled hydroxy amine substituted with an alkynyl group (e.g., O-(4-ethynylphenyl)-hydroxylamine) providing an oxime labeled compound comprising an alkynyl group; and contacting the oxime labeled compound comprising an alkynyl group with a solid support conjugated to a triazene group under conditions such that the meta or para alkynyl group reacts with the triazene group on the solid support to form a triazole linkage providing a solid support conjugated to the oxime labeled compound; washing the solid support to remove unreacted material from the sample providing a purified solid support conjugated to the oxime labeled compound; and contacting the purified solid support conjugated to the oxime labeled compound to conditions providing a composition with a purified compound comprising a compound or peptide released from the solid support.

In certain embodiments, the conditions are acid conditions, e.g., exposure to trifluoroacetic acid.

In certain embodiments, the methods further comprise determining the molecular weight or exact mass of the compound or peptide.

In certain embodiments, the methods further comprise separating the composition with a purified compound into two or more compounds. In certain embodiments, separating is by chromatography. In certain embodiments, for any of the methods disclosed herein the methods further comprise separating a purified, cleaved, or isolated peptide composition or compound composition into two or more peptides or compounds. In certain embodiments, separating is by chromatography, e.g., adsorption and/or size-based separations, or filtration through a gel.

In certain embodiments, methods comprise obtaining a sample from a subject; contacting the sample comprising a protein in a sample with a protease providing a cleaved protein; contacting the cleaved protein with an alpha alkynyl ketone providing a formamide and chalcone derivative; contacting the formamide and chalcone derivative with a labeled hydroxy amine providing an oxime labeled cleaved protein in the sample; and purifying the oxime labeled cleaved protein by chromatography.

In certain embodiments, this disclosure relates to methods of purifying a compound or peptide with a N-alkylated imidazole ring such as methyl histidine in a sample comprising, contacting the compound or peptide with an alpha alkynyl ketone providing a formamide and chalcone derivative and contacting the sample with a solid support conjugated to a compound comprising an hydroxy amine such that the compounds with a formamide and chalcone derivative in the sample reacts with the hydroxy amine providing oxime compound immobilized to the solid support; washing the solid support to remove unreacted material from the sample providing a purified oxime compound immobilized to the sold support. In certain embodiments, the method further comprises and exposing the purified compound immobilized to the solid support to conditions releasing a purified compound or peptide.

In certain embodiments, this disclosure relates to an oxime labeled compound or solid support such as a particle conjugated with a compound having a N-methyl formamide derivative of following formula,

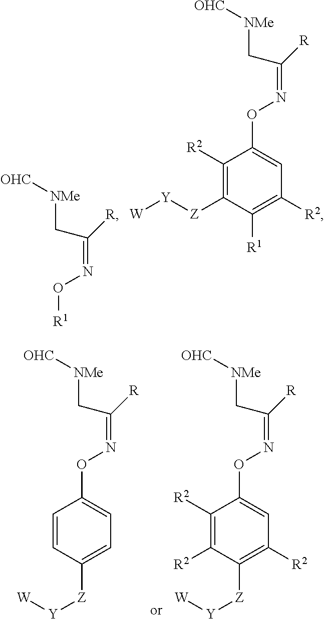

or derivative or salt thereof wherein,
   W is a solid support such as a particle or a label;
   Y is O, NH, or S;
   Z is a linking group;
   R is a peptide, nucleic acid, nucleobase polymer, compound or other biological molecule, i.e., contained an N-alkylated imidazole ring such as methyl histidine; or R is alkyl, aryl, carbocyclyl, or heterocyclyl, wherein R is optionally substituted with one or more substituents;
   R¹ is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R¹ is optionally substituted with one or more substituents, or conjugated to a label or a solid support through a linking group;
   R² is individually and independently at each occurrence are individually and independently at each occurrence hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R² is optionally substituted with one or more substituents, —Z—W, or —Z—Y—W, or two R²s and the attached atoms come together to form an aromatic or non-aromatic ring that is optionally substituted with one or more substituents or —Z—W, or —Z—Y—W.

In certain embodiments, this disclosure relates to a compound or material comprising or coated with or conjugate to chemical arrangements disclosed herein.

In certain embodiments, a material, solid support, or particle further comprises or is coated with or conjugated to a hydrophilic polymer.

In certain embodiments, this disclosure contemplates that the solid support is a magnetic material, e.g., magnetic bead, and purifying is capturing the magnetic bead with a magnetic field thereby separating the magnetic bead and contents thereof from a solution or mixture.

### EXAMPLES

### Labeling N-Methylhistidine (Hme)-Alanine-Phenylalanine (HmeAF) Peptide

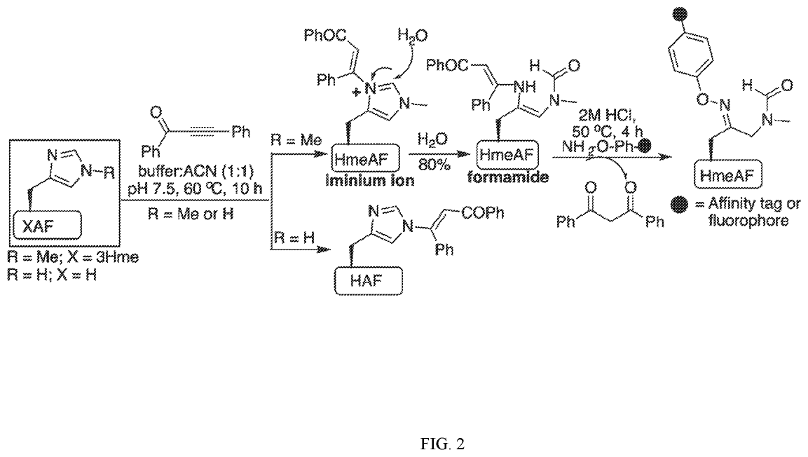

HmeAF is reacted with 1,3-diphenylprop-2-yn-1-one providing a formamide and chalcone derivative followed by a reaction O-phenyl hydroxy amine substituted with an affinity tag or fluorophore providing an O-phenyl oxime labeled peptide (FIG. 2). A 2 mL glass vial is equipped with a stir bar, and 2.34 μmol of HmeAF, 11.7 μmol of 1,3-diphenylprop-2-yn-1-one (5 eq), and 4.67 μmol of potassium carbonate (2 eq) were added. The reaction was diluted with acetonitrile and water to give a 1:1 mixture and total volume of 392 μL. The reaction was stirred overnight at 50° C. in an oil bath. To the same reaction, 131 L of 2 M hydrochloric acid (1:3, 2 M HCl: reaction mixture) and 23.4 μmol of O-benzylhydroxylamine hydrochloride (10 eq) were added. The reaction was left to stir for an additional three hours at room temperature.

1. A method of forming an oxime compound, comprising contacting a compound containing an N-alkylated imidazole ring with 1,3-diphenylprop-2-yn-1-one or derivative providing an N-methyl formamide and contacting the N-methyl formamide with a hydroxy amine providing an oxime compound.

2. The method of claim 1, wherein the compound containing an N-alkylated imidazole ring is methyl histidine or peptide comprising the same.

3. The method of claim 1, wherein contacting the compound containing the N-alkylated imidazole ring with 1,3-diphenylprop-2-yn-1-one is in a solution of acetonitrile and water.

4. The method of claim 3, wherein the solution of acetonitrile and water is at a pH of between 6 and 8.

5. The method of claim 3, wherein the solution of acetonitrile and water is at a temperature of between 50 degrees C. and 70 degrees C.

6. The method of claim 1, wherein contacting the formamide derivative with the hydroxy amine is in an aqueous solution at a pH of less than 2.

7. The method of claim 1, wherein contacting the formamide derivative with the hydroxy amine is at a temperature of between 40 degrees C. and 60 degrees C.

8. The method of claim 1, wherein the oxime comprises a label.

9. The method of claim 8, wherein the label is an aromatic molecule, a fluorescent dye, a ligand, biotin, a receptor, an antibody, or an antigen.

10. A peptide, nucleic acid, nucleobase polymer, or compound comprising the oxime made by the process of claim 1.

11. A solid support conjugated to a peptide, nucleic acid, nucleobase polymer, or compound comprising the oxime made by the process of claim 1.
